(19) United States
(12) Patent Application Publication
(10) Pub. No.: US 2006/0155188 A1
(43) Pub. Date: Jul. 13, 2006

(54) Method and system for determination of vessel tortuousity

(76) Inventors: Alan M. Walczak, West Seneca, NY (US); Kenneth R. Hoffmann, Williamsville, NY (US); Petru M. Dinu, Buffalo, NY (US); Sebastian Schafer, Buffalo, NY (US)

Correspondence Address: HODGSON RUSS LLP, ONE M & T PLAZA, SUITE 2000, BUFFALO, NY, 14203-2391, US

(21) Appl. No.: 11/286,724

(22) Filed: Nov. 23, 2005

Related U.S. Application Data
(60) Provisional application No. 60/630,687, filed on Nov. 24, 2004.

(51) Int. Cl. A61B 5/05

(52) U.S. Cl. 600/421

(57) ABSTRACT

A method for determining suitability of a device, such as a stent, for delivery through a vessel comprises the steps of generating a model representing positions of the device within the vessel, and evaluating the model to ascertain a degree of risk associated with delivery of the device through the vessel. The model may be generated based upon data describing the vessel and data describing the device. In a preferred embodiment, a color-coded image of the vessel is generated to report local vessel tortuousity relative to the device.

### CROSS-REFERENCE TO RELATED APPLICATIONS

The present application claims benefit of U.S. Provisional Patent Application No. 60/630,687 filed Nov. 24, 2004, the entire disclosure of which is hereby incorporated herein by reference.

### STATEMENT REGARDING FEDERALLY SPONSORED RESEARCH

This work was supported by Grant No. RO1 EB002916 from the National Institutes of Health. The United States Government has certain rights in the invention.

### FIELD OF THE INVENTION

The invention relates generally to the field of endovascular interventional procedures, and more specifically to a method and system for assessing vessel tortuousity with respect to an endovascular device in preparation for delivery of the device through the vessel.

### BACKGROUND OF THE INVENTION

Clinicians currently decide which size stent to use based on diameter and length of the lesion being treated. However, the vessel path to the intervention site is often tortuous, as in the case of the carotid and vertebral arteries. Because of the tortuousity, clinicians experience difficulties and complications in about 10% of cases. Consequently, there is a need for a method and system that will enable clinicians to determine whether a particular endovascular device, e.g. a stent, will be able to pass through the vessel to the intervention site, and to ascertain ahead of time the vessel locations which present the highest degree of risk for delivery complications.

### SUMMARY OF THE INVENTION

It is thus an object of the present invention to provide a method and system that allow clinicians to determine whether a device will pass through a vessel before delivery is attempted.

It is another object of the present invention to provide a method and system for mapping degrees of risk along the vessel so that clinicians may easily identify locations where difficulties are more likely to be encountered.

It is a further object of the present invention to provide a method and system that enable clinicians to compare tortuousity related risks associated with differently sized devices, e.g. stents of different lengths and/or different undeployed diameters, to select the most suitably sized device for an intervention procedure.

It is yet a further object of the present invention to provide a method and system that will reduce the number of attempts with endovascular devices which might result in complications, thereby reducing mortality, morbidity, and cost associated with endovascular interventional procedures.

In furtherance of these and other objects, a method and system for determining vessel tortuousity relative to a given endovascular device is described herein. A method in accordance with an embodiment of the present invention generally comprises the steps of generating a model representing positions of the stent within the vessel, and evaluating the model to ascertain a degree of risk associated with delivery of the stent through the vessel. The method may comprise the further step of generating a color-coded image of the vessel for communicating the degree of risk.

The present invention is also embodied in a method generally comprising the steps of reading vessel data describing the vessel, reading device data describing the device, generating a model representing positions of the device along a travel path through the vessel based on the vessel data and the device data, and evaluating the model to ascertain a degree to which the device protrudes from the travel path and/or deforms when the device is positioned at a corresponding point along the travel path. The vessel data may be provided by a separate image acquisition system. The device data may be provided by user input or a device database. The model is preferably evaluated for a plurality of points each corresponding to a different position of the device along the travel path. The travel path may correspond to a centerline path running through the center of the vessel, a path of a guidewire inserted through the vessel, or a “free floating” path determined by vessel boundaries and physical properties of the device. The degree to which the device protrudes from the travel path and/or deforms when the device is positioned at a point along the travel path is preferably reported by generating a color-coded image of the vessel, wherein points along the vessel are colored depending upon the local degree of protrusion and/or deformation.

A system configured in accordance with the present invention to determine tortuousity of a vessel with respect to a device generally comprises a computer system storing programming code for generating a model representing positions of the device along a travel path through the vessel based on data describing the vessel and the device, and programming code for evaluating the model to ascertain a degree to which the device protrudes from the travel path and/or deforms when the device is positioned at a corresponding point along the travel path. Preferably, the programming code for evaluating the model causes the model to be evaluated for a plurality of points each corresponding to a different position of the device along the travel path. In a preferred embodiment, the computer system stores programming code for reporting the degree on an output device, such as a display or printer, which may be included in the computer system. In a preferred embodiment, the programming code for reporting the degree generates a color-coded image of the vessel on a display, wherein different colors in the image represent different degrees to which the device protrudes from the travel path and/or deforms.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The nature and mode of operation of the present invention will now be more fully described in the following detailed description taken with the accompanying drawing figures, in which:

FIG. 1 is a schematic block diagram of a system formed in accordance with an embodiment of the present invention;

FIG. 2 is a flowchart generally illustrating a method in accordance with an embodiment of the present invention;

FIGS. 3A through 3C are generated images of a vessel from three different viewpoints, wherein the vessel is color-coded according to an embodiment of the present invention to indicate localized degrees of vessel tortuousity relative to passage of a given stent;

FIGS. 4A through 4C are generated images of a vessel, all from the same viewpoint, wherein the vessel is color-coded according to an embodiment of the present invention to indicate localized degrees of vessel tortuousity relative to passage of three differently-sized stents, respectively;

FIG. 5 is a flowchart illustrating program flow according to a first example embodiment of the present invention;

FIG. 6 is a flowchart detailing a deviation routine associated with the program flow of FIG. 5;

FIG. 7 is a schematic view of a vessel and stent model showing virtual positions of a stent through the vessel in accordance with the first example embodiment;

FIG. 8 is an enlarged schematic view of the circled region “A” in FIG. 7;

FIG. 9 is a flowchart illustrating program flow according to a second example embodiment of the present invention;

FIG. 10 is a flowchart detailing a distance routine associated with the program flow of FIG. 9;

FIG. 11 is a schematic view of a vessel and stent model showing virtual positions of a stent through the vessel in accordance with the second example embodiment;

FIG. 12 is an enlarged schematic view of the circled region “A” in FIG. 11;

FIG. 13 is a flowchart illustrating program flow according to a third example embodiment of the present invention;

FIG. 14 is a flowchart detailing a cone routine associated with the program flow of FIG. 13;

FIG. 15 is a schematic view of a vessel and stent model showing a virtual position of a stent within the vessel and alternate position cones associated with proximal and distal segments of the stent in accordance with the third example embodiment;

FIG. 16 is a view facing a base of circle of an alternate position cone shown in FIG. 15;

FIG. 17 is a flowchart illustrating program flow according to a fourth example embodiment of the present invention;

FIG. 18 is a flowchart detailing a segment adjustment routine associated with the program flow of FIGS. 17 and 22;

FIGS. 19A through 19D are a series of schematic views of a vessel and stent model showing virtual positioning of a stent within a vessel in accordance with the fourth example embodiment;

FIG. 20 is a schematic view of a vessel and stent model showing advancement of the stent to a next location in the vessel in accordance with the fourth example embodiment;

FIG. 21 is an enlarged view of the stent as positioned according to FIG. 19D, annotated to indicate bend angles between stent segments;

FIG. 22 is a flowchart illustrating program flow according to a fifth example embodiment of the present invention;

FIGS. 23A through 23F are a series of schematic views of a vessel and stent model showing virtual positioning of a stent within a vessel in accordance with the fifth example embodiment;

FIG. 24 is a schematic view of a vessel and stent model showing advancement of the stent to a next location in the vessel in accordance with the fifth example embodiment;

FIG. 25 is an enlarged view of the stent as positioned according to FIG. 23F, annotated to indicate bend angles between stent segments;

FIG. 26 is a flowchart illustrating program flow according to a sixth example embodiment of the present invention; and

FIG. 27 is a schematic view of a vessel and stent model showing a virtual position of a stent within a vessel in accordance with the sixth example embodiment.

### DETAILED DESCRIPTION OF THE INVENTION

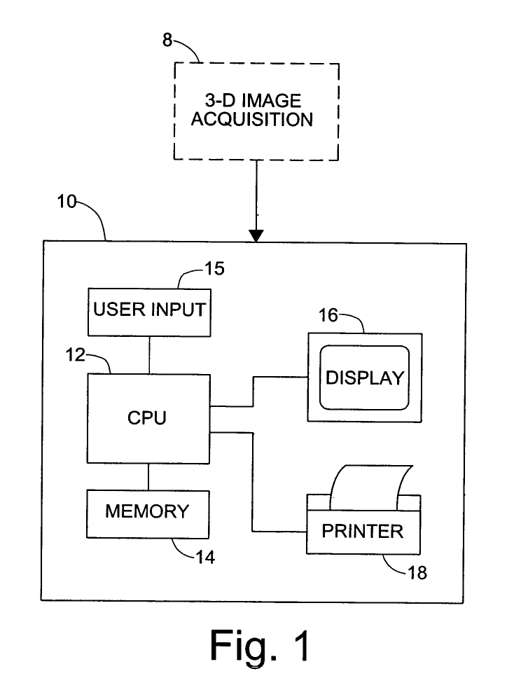

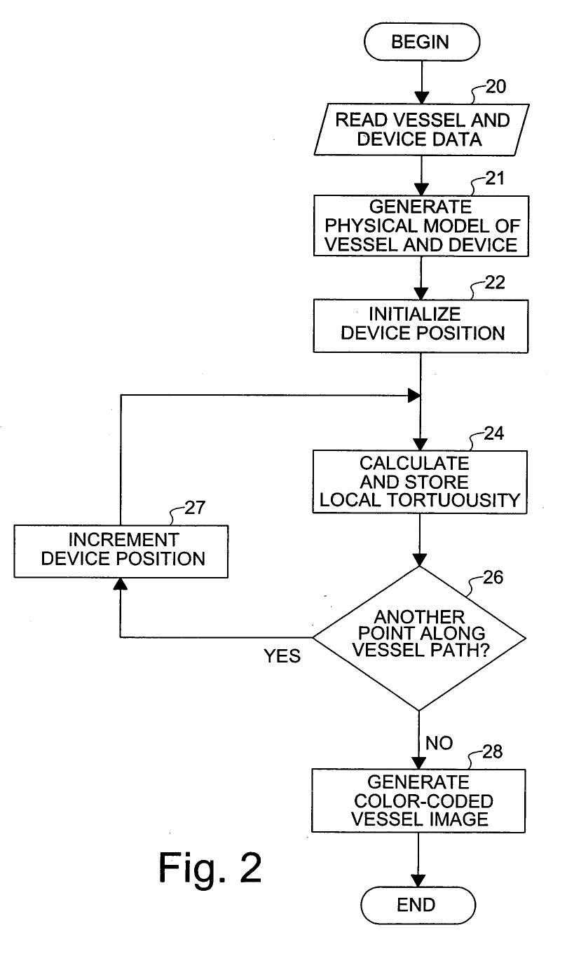

FIGS. 1 and 2 respectively depict a system and method embodying the present invention. FIG. 1 shows a computer system 10 comprising a central processing unit (CPU) 12 in communication with a memory 14 storing data and programming code for execution of a method in accordance with FIG. 2. The depicted computer system 10 further comprises a user input device 15, a display 16, and a printer 18 each connected to CPU 12. The configuration of computer system 10 is of course subject to variation, and may take the form of a personal computer system, a workstation connected to a network server, or other forms. Similarly, it will be understood that memory 14 may take a variety of forms, including but not limited to a hard drive, a removable memory device, or a remote memory device.

Also shown in FIG. 1, but not itself part of computer system 10, is a three-dimensional image acquisition system 8 for acquiring image data describing vascular structure in a patient. Image acquisition system 8 may be, for example, a computed tomography angiography (CTA) imaging system, a magnetic resonance imaging (MRI) system, a rotational angiography imaging system, a biplane imaging system, another suitable imaging system, or any combination of imaging systems. Image acquisition system 8 acquires data that describe a vessel and enable determination of further data that also describe the vessel; all such data acquired directly by the image acquisition system 8 or derived from the acquired data are referred to herein as “vessel data.” Vessel data may include, without limitation, parameters for defining a three-dimensional vessel centerline, vessel diameters at various locations along the vessel, and/or vertices describing an inner wall surface of the vessel. The image acquisition system 8 may also be used to acquire guidewire data that describe a guidewire inserted through the vessel during surgery as part of a stent delivery system, and which enable determination of further guidewire data that also describe the guidewire. For example, guidewire data may include parameters for defining a three-dimensional path of a guidewire through the vessel.

Turning now to FIG. 2, a flowchart is provided that represents the functions performed by programming code stored in memory 14 and executed by computer system 10. These functions, taken together, perform a method for determining “tortuousity” of a vessel relative to a device, such as a stent, catheter, or other medical or diagnostic device that is to be moved through the vessel. In the context of the present specification, the term “tortuousity” generally means a degree to which the vessel includes changes in direction, and the severity and sharpness of such changes in direction. Thus, in determining tortuousity of a vessel relative to a given device, a degree to which the device protrudes from a defined travel path through the vessel (i.e. a centerline through the vessel) is indicative. Intuitively, imagine a bus making a sharp turn on a city street: the bus will protrude to some extent from its lane of traffic as it makes the turn (contrast this with the bus traveling along a long, straight stretch of highway). If the device is capable of bending or deforming as it travels through the vessel, then a degree to which the device bends or deforms within the vessel provides another indicator for determining tortuousity of the vessel relative to the device. Intuitively, imagine a double tractor-trailer having two trailer segments joined end-to-end by a hitch: the trailer segments will rotate relative to one another about the hitch point to “deform” the vehicle as the vehicle makes a turn (again, contrast this with the double tractor-trailer traveling along a long, straight stretch of highway).

The first step at block 20 in FIG. 2 is to read vessel and device data. As described above, the vessel data are provided directly by image acquisition system 8 and/or derived from data provided by image acquisition system 8. Where the device is designed for travel along a guidewire inserted through the vessel, then guidewire data may also be read. Guidewire data may be provided directly by image acquisition system 8 and/or derived from data provided by image acquisition system 8. The device data may be physical parameters of the device. For example, in the case of a stent, the device data may include a length of the stent, a diameter of the stent, and data representing deformation capability of the stent. The device data may be stored by memory 14, preferably as part of a database containing data corresponding to a plurality of devices of a generic type, such as stents. Alternatively, the computer system 10 may prompt a user to input device data using input device 15.

The vessel data and device data are used to generate a digital physical model of the vessel and device in accordance with block 21. With regard to the vessel, the physical model generally represents a travel path along which the device is located at different positions through the vessel, and a boundary corresponding to an inner wall surface of the vessel. Where a guidewire is used, the travel path will be determined, at least to a large extent, by a path of the guidewire through the vessel. The travel path may be represented by a parametric curve in three dimensions (the set of points x=f(t), y=g(t), z=h(t) where t is the parameter corresponding to points along the travel path). With regard to the device, the physical model generally represents size and shape of the device, and may represent joint positions and maximum bending angles to simulate deformation capability, as will understood in the examples that follow. In an effort to simplify physical modeling of the device, particularly where the device is a stent, the vessel boundary in the model may be adjusted by reducing the virtual vessel's internal diameter by an amount equal to the stent diameter, whereby the stent diameter may be disregarded and the device may be represented by a straight line segment or a series of straight line segments connected end-to-end by one or more joints.

The next step is to model an initial position or range of positions of the device at an initial point along the travel path, as indicated by block 22. Then, the model is evaluated to ascertain a degree to which the device protrudes from the travel path and/or deforms when the device is positioned at that point along the travel path. As described above, this degree is indicative of vessel tortuousity at that point along the travel path in relation to the device. The degree may be calculated quantitatively and stored as a tortuousity value local to that point along the travel path in accordance with block 24. Various specific approaches to performing this step are described with respect to the examples that follow.

Flow then continues to a decision 26 to determine if there is another point along the travel path. If so, the device is moved in the model to the next point along the travel path, and its position or range of positions when located at the next point is determined in block 27. Preferred specific methodologies for repositioning the virtual device are described in the examples below, and will depend on the physical model of the device (e.g. whether the device is rigid or deformable). Flow then branches back to block 24 for calculation of local tortuousity.

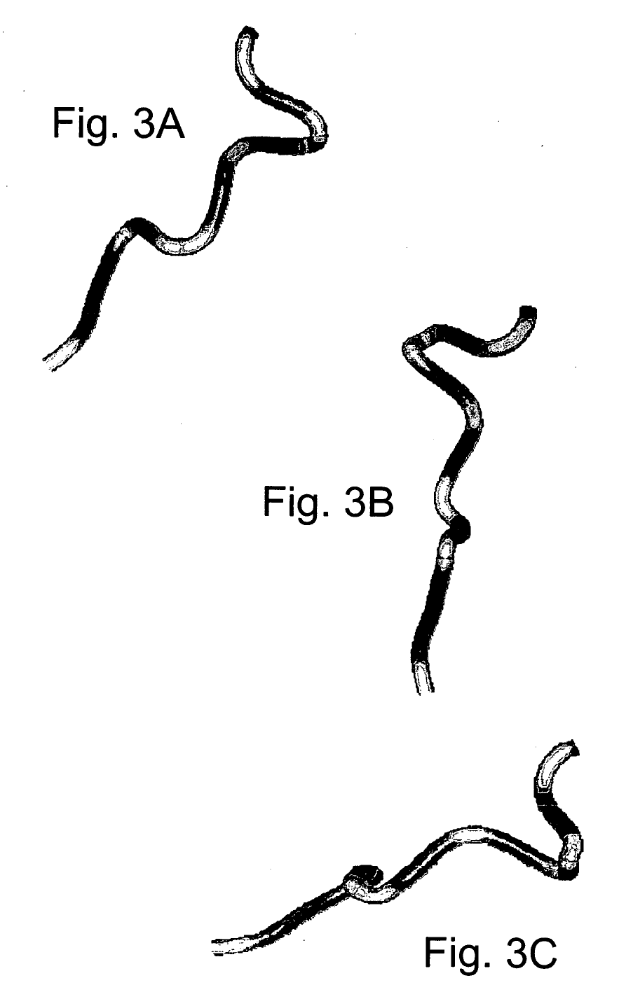

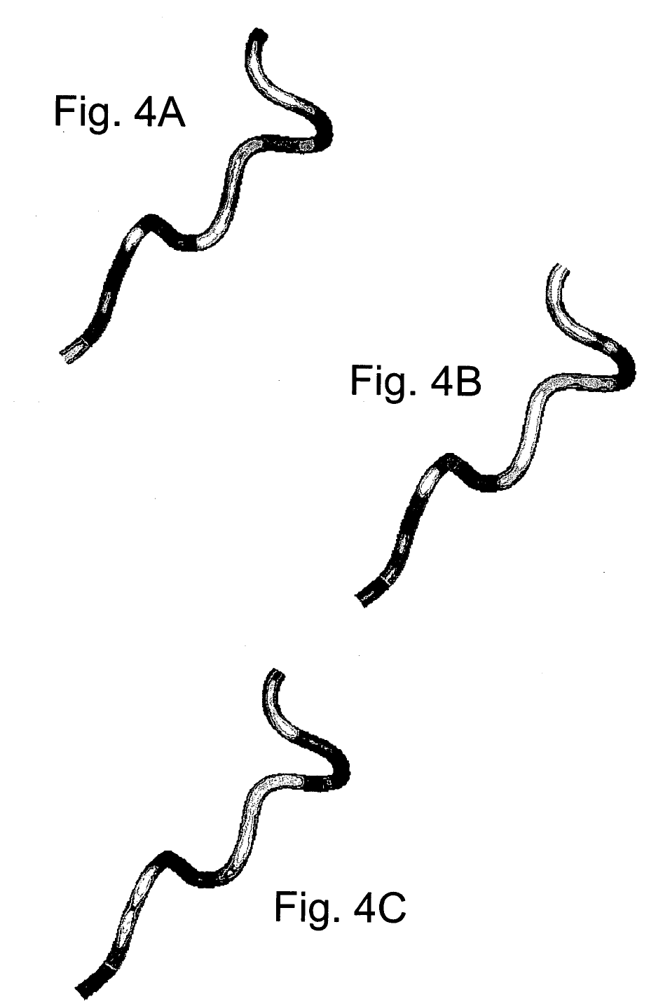

Once the last point along the travel path has been reached and evaluated, decision 26 returns “NO” and flow proceeds to block 28. In this step, the tortuousity values are reported by generating a color-coded image of the vessel, wherein different colors in the image represent different degrees to which the device protrudes from the travel path and/or deforms at various locations along the vessel. It has been found suitable to assign a differentiating color to each of several ranges of calculated tortuousity values. By way of example, green may indicate a relatively low degree of tortuousity, yellow a relatively moderate degree of tortuousity, and red a relatively high degree of tortuousity. Examples of such images are provided in FIGS. 3A-3C, which show the same vessel rendered from three different viewpoints with respect to the same stent. Further examples are provided in FIGS. 4A-4C, which show the same vessel from the same viewpoint for three differently-sized stents, wherein the stent size becomes larger progressing from FIG. 4A to FIG. 4C. As will be understood from looking at FIGS. 4A-4C, as the stent become larger, tortuousity of the vessel increases relative to the stent, as expected. Although the flow diagram of FIG. 2 shows that the color-coded image is generated after all local tortuousities have been calculated, the image may of course be generated stepwise as each local tortuousity value is calculated.

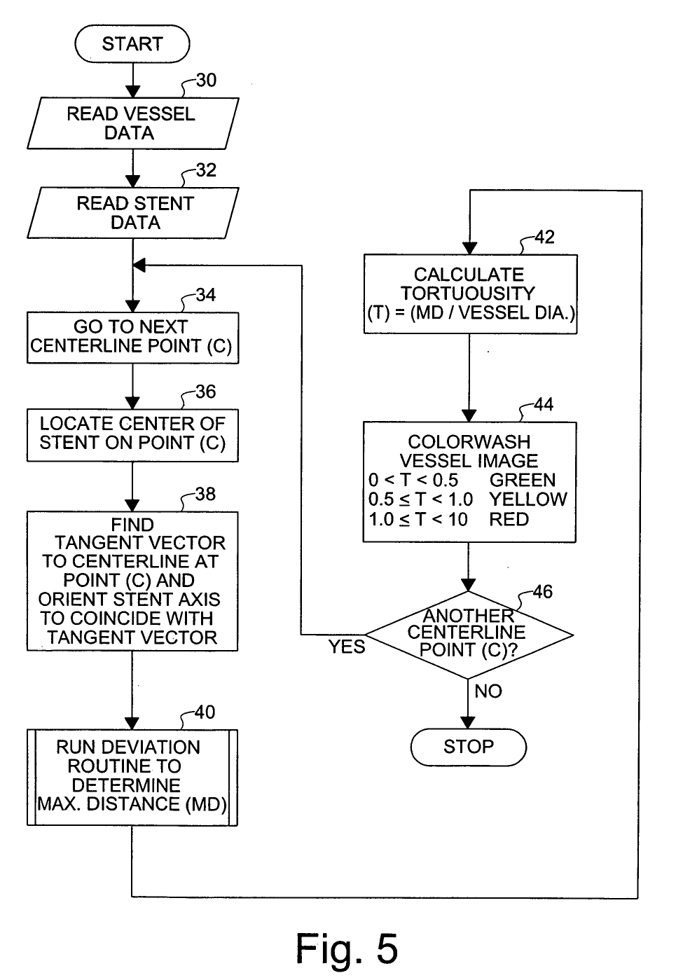

The foregoing description is intended to describe the present invention in a broad manner, so that the steps of the invention are not construed narrowly as requiring a particular mathematical approach or assumption for generating the model, positioning the device, or calculating the degree of tortuousity. To further illustrate how the present invention may be practiced, several specific examples are described below in conjunction with FIGS. 5-27. The claims, however, should not be construed as requiring any particular approach or assumption described in the specific examples.

### EXAMPLE 1

### Rigid Stent Alone Vessel Centerline Path

Reference is made to FIGS. 5-8 of the drawings. In this example, a stent 66 of length SL is assumed to be rigid, and the center of the stent is always positioned on a centerline curve 64 extending through the vessel 60 and centered with respect to vessel boundary surface 62.

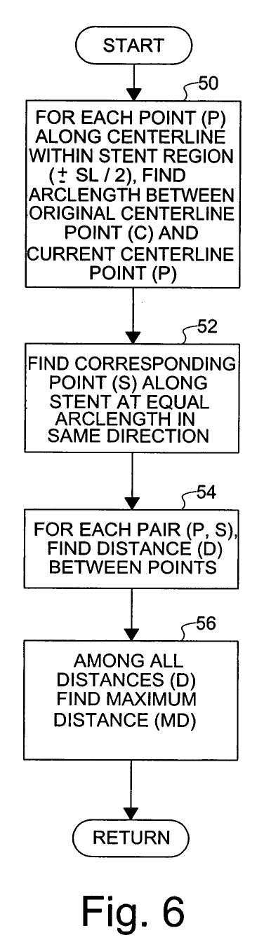

As shown in the flow diagram of FIG. 5, vessel data and stent data, such as the stent length (SL) and stent diameter, are read in blocks 30 and 32, respectively. The next vessel centerline point C (or initial point, if it is the first to be evaluated) is determined in block 34, and the center of stent 66 is located at centerline point C in accordance with block 36. Then, in block 38, the tangent vector to the centerline curve at point C is determined (this is the first derivative of the centerline curve at point C, analogous to velocity vector where parameter is time), and the stent axis is oriented to coincide with the direction of the tangent vector. A deviation routine is then executed as indicated by block 40 to determine a “maximum distance” MD associated with the position of the stent. The deviation routine is shown in detail in FIG. 6, and can be understood with further reference to FIG. 8. Under block 50, a series of points P along centerline 64 within the region of stent 66 are defined, and for each point, the arclength along the centerline curve between the original centerline point C and the particular point P is found. Then, in block 52, a corresponding point S at an equal arclength along stent 66 in the same direction is determined, resulting in a series of corresponding pairs of points P and S. For each of these pairs, a distance D between the points P and S is determined pursuant to block 54. The deviation routine then determines the maximum distance MD from among all of the distances D in block 56 and returns this value.

Returning now to block 42 in FIG. 5, a tortuousity value T associated with centerline point C is calculated as the ratio of the maximum distance MD to the vessel diameter at centerline point C. A colorwash function is carried out in block 44 to color a corresponding location on a digital image of the vessel based upon the local tortuousity value T. In the present example, the vessel location corresponding to centerline point C is colored green if T is less than 0.5, yellow if T is equal to or greater than 0.5 and less than 1.0, and red if T is equal to or greater than 1.0 and less than 10.0. As mentioned above, this step may be performed after T has been calculated for all centerline points C, or after each calculation of T as shown in FIG. 5.

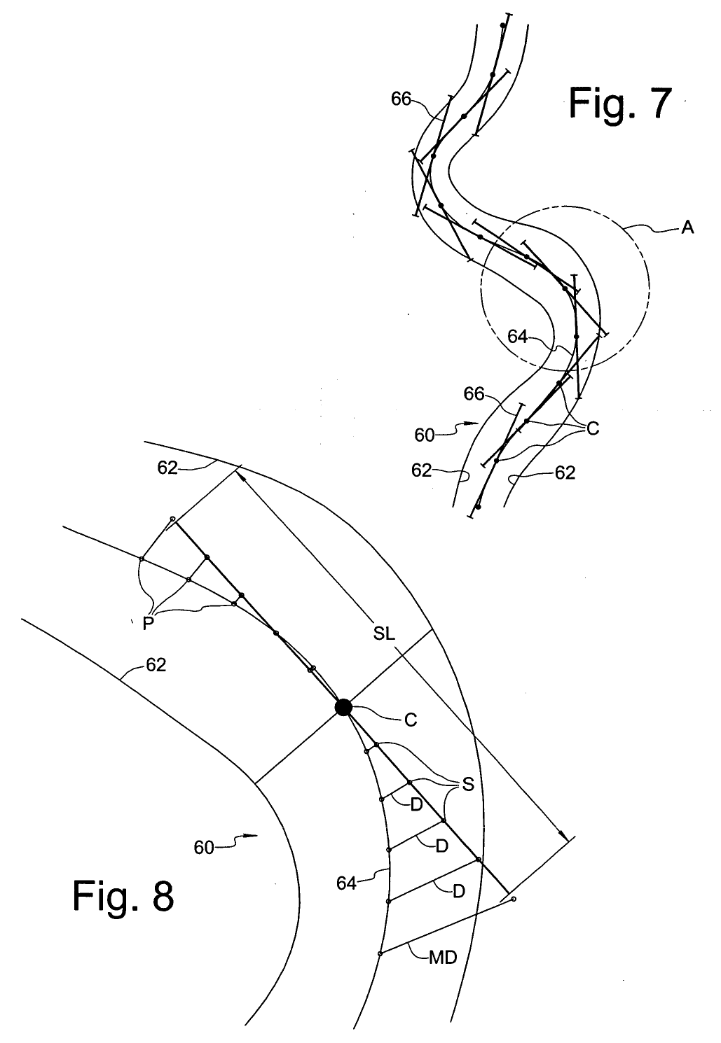

Decision 46 determines if another centerline point C exists. If so, flow reverts to block 34 to repeat the procedure of positioning the stent 66 and evaluating the stent position to determine tortuousity T at the next centerline point C. FIG. 7 shows the stent positioned at various points C along centerline 64. If there are no further centerline points C, the program is completed.

### EXAMPLE 2

### Rigid Stent at Boundary-Limited Positions

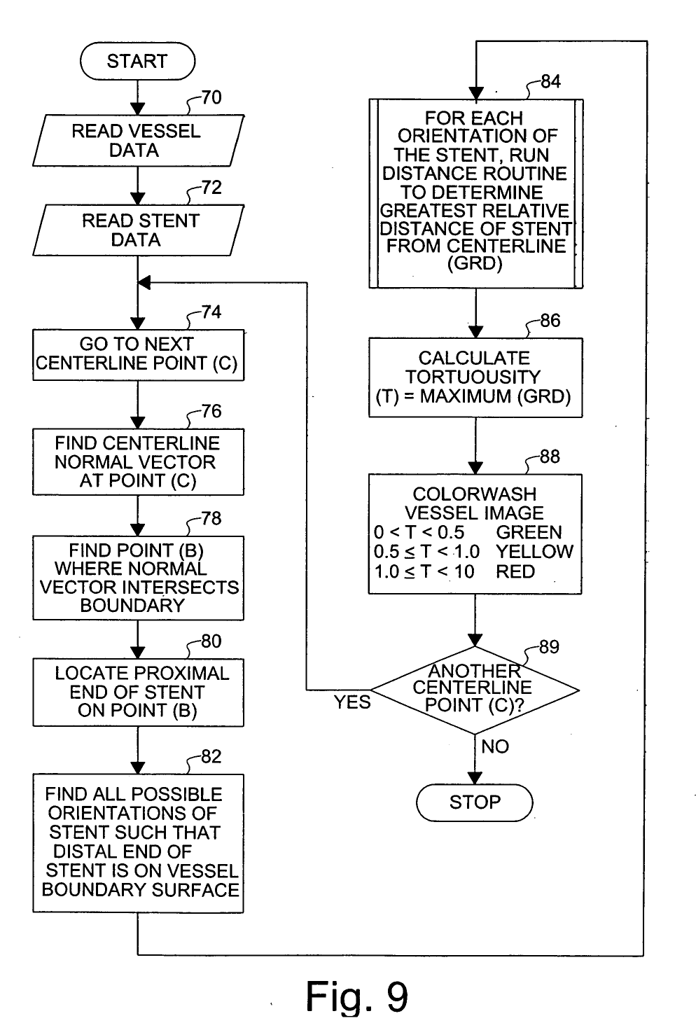

Reference is made to FIGS. 9-12 of the drawings. In this example, a stent 66 of length SL is assumed to be rigid, and a proximal end of the stent is always located at a point on the vessel boundary 62.

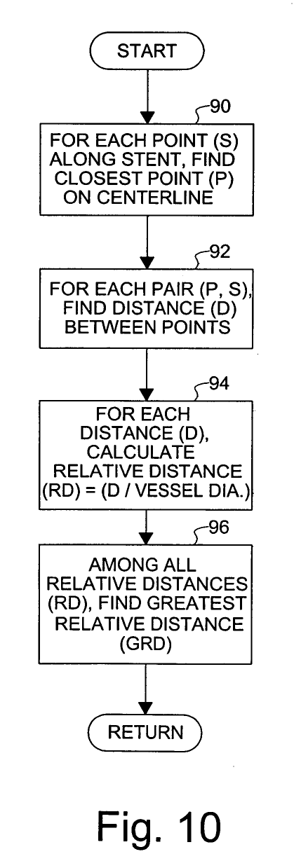

As shown in the flow diagram of FIG. 9, vessel data and stent data, such as the stent length (SL) and stent diameter, are read in blocks 70 and 72, respectively. The next vessel centerline point C (or initial point, if it is the first to be evaluated) is determined in block 74, and the normal vector to the centerline curve at point C is determined (this is the second derivative of the centerline curve at point C, analogous to acceleration vector where parameter is time) in block 76. The point B where the normal vector intersects boundary 62 (see FIG. 12) is found in block 78, and a proximal end of stent 66 is located at point B in block 80. Once the proximal end has been placed at point B, all possible orientations of the stent wherein a distal end of the stent is on the vessel boundary 62 are found in block 82. For each of these possible orientations, a distance routine is executed according to block 84 to determine a greatest relative distance of a point S on the stent to a closest corresponding point P on centerline 64. The distance routine called in block 84 is shown in the flowchart of FIG. 10. For each point S along the stent, the closest point P on centerline 64 is found in block 90, thereby providing a series of corresponding pairs of points P and S. For each of these pairs, a distance D between the points P and S is determined pursuant to block 92. Then, each distance D is converted to a relative distance RD in block 94 by dividing the distance D by the local vessel diameter. Finally, according to block 96, the distance routine finds the greatest relative distance (GRD) from among all the computed relative distances RD, and returns this value.

Returning now to block 86 in FIG. 9, a tortuousity value T is deemed to be the maximum of all the GRD values. This value is assigned to the vessel centerline point C′ closest to the distal end of stent 66. A colorwash function is carried out in block 88 to color a corresponding location on a digital image of the vessel based upon the local tortuousity value T. In the present example, the vessel location corresponding to centerline point C′ is colored green if T is less than 0.5, yellow if T is equal to or greater than 0.5 and less than 1.0, and red if T is equal to or greater than 1.0 and less than 10.0. This step may be performed after T has been calculated for all centerline points under consideration, or after each calculation of T as shown in FIG. 9.

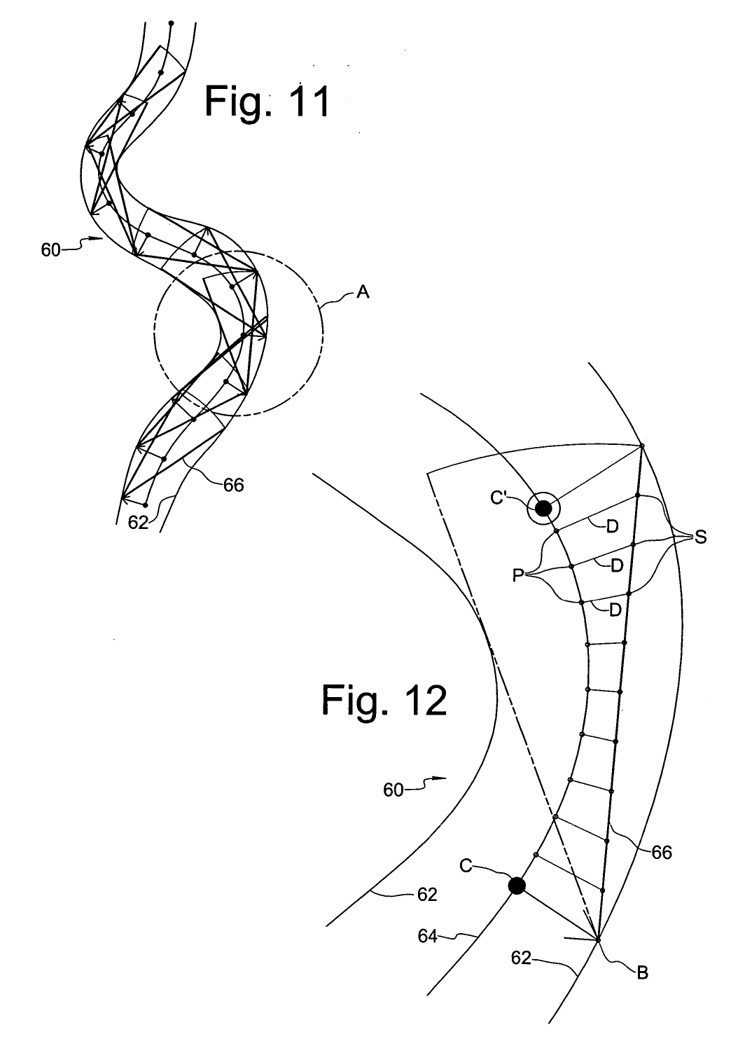

Decision 89 determines if another centerline point C exists. If so, flow reverts to block 74 to repeat the procedure of positioning the stent 66 and evaluating the stent positions to determine tortuousity T. FIG. 11 shows stent position ranges through a portion of vessel 60 using an approach according to the present example. If there are no further centerline points C, the program is completed.

### EXAMPLE 3

### Single Joint Stent Along Vessel Centerline Path

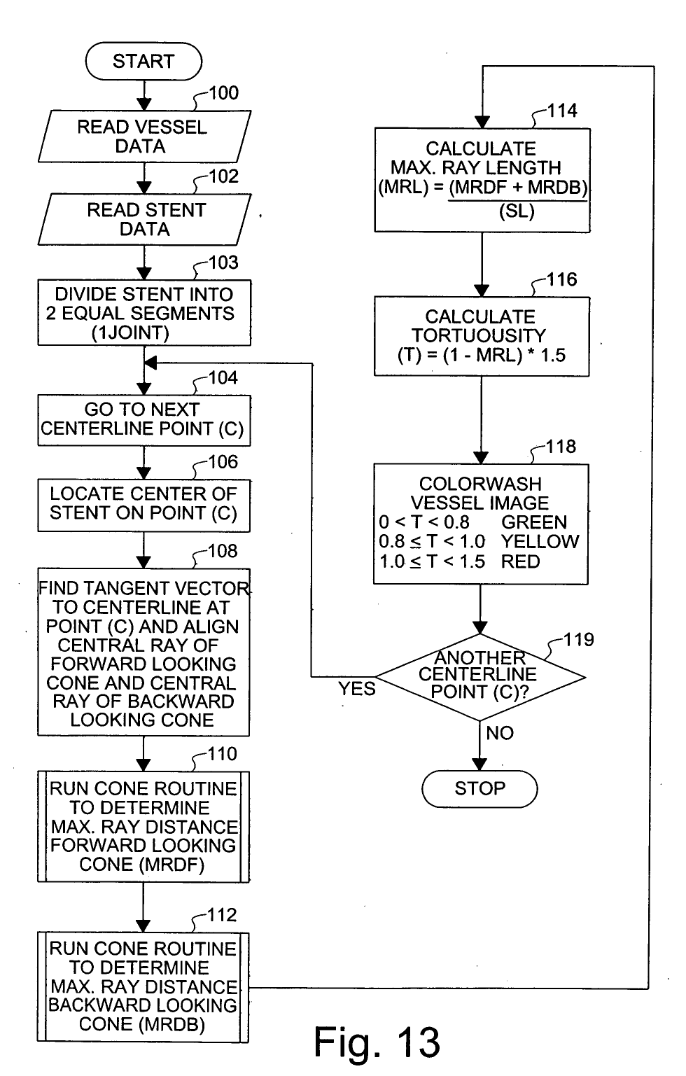

Reference is made to FIGS. 13-16 of the drawings. In this example, a stent of length SL is assumed to comprise two segments 66a and 66b of equal length SL/2 connected end-to-end by a center joint 67, and the center joint of the stent is always positioned on a centerline curve 64 extending through the vessel 60.

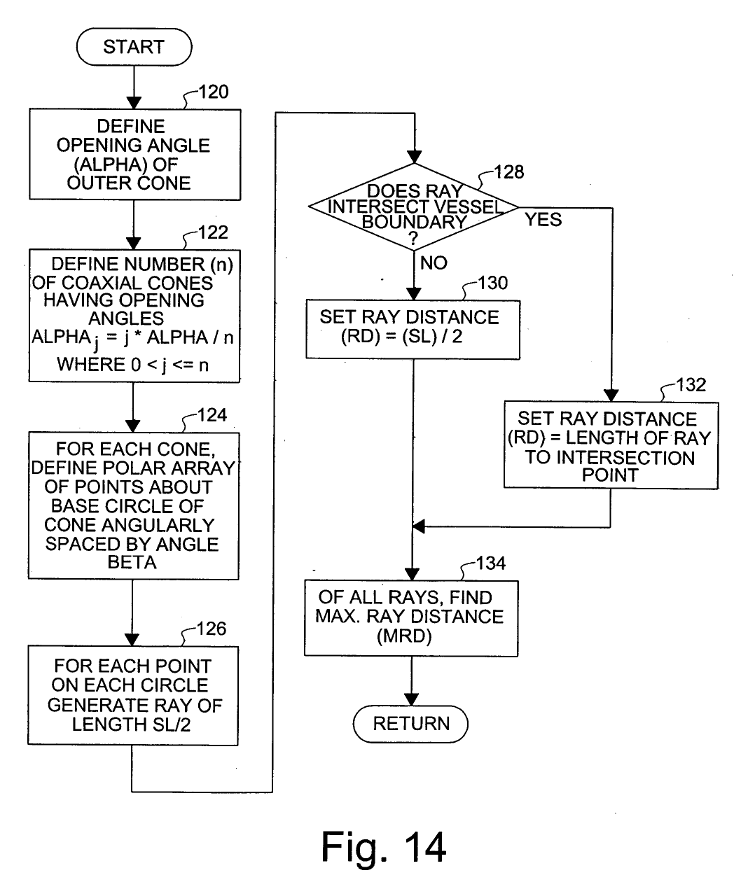

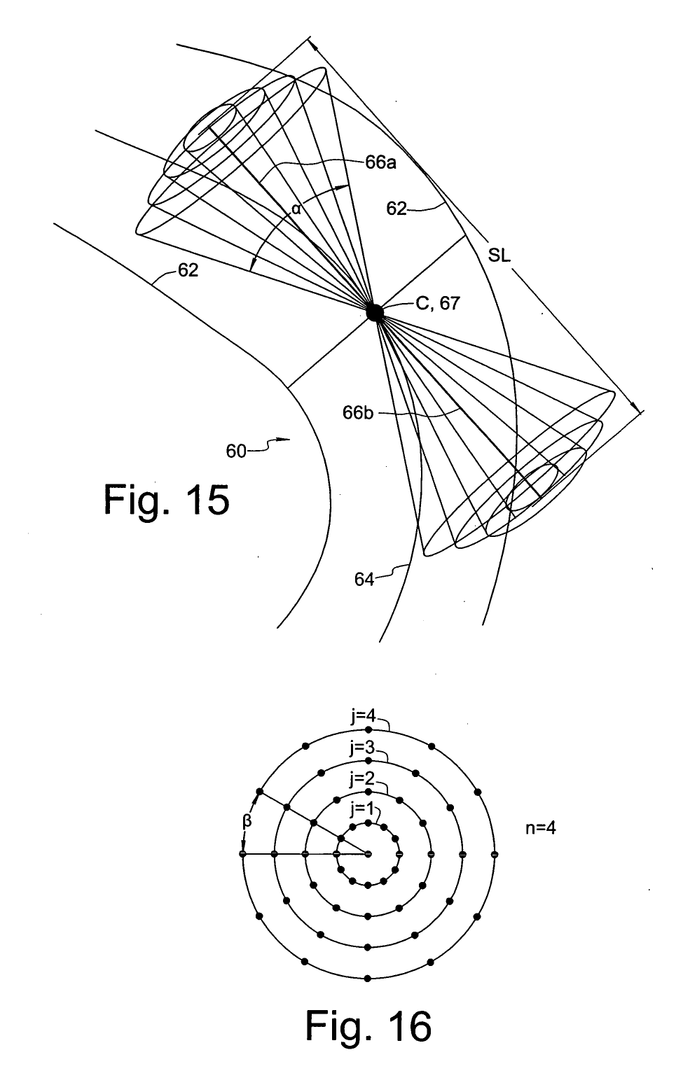

The flow diagram of FIG. 13 begins with blocks 100 and 102, wherein the vessel data and stent data are read as input data. Then, in block 103, the stent is divided into two equal segments 66a and 66b by introducing a joint 67 midway along the length of the stent, whereby the stent segments are assumed to be capable of pivoting about the center of joint 67 relative to one another as if connected by a ball joint. The next vessel centerline point C (or initial point, if it is the first to be evaluated) is determined in block 104, and the center of the stent (that is, joint 67) is located at centerline point C in accordance with block 106. Then, in block 108, the tangent vector to the centerline curve at point C is determined, and the direction of the tangent vector defines a central ray of a forward looking cone representing a range of alternate positions of distal segment 66a, and a central ray of a backward looking cone representing a range of alternate positions of proximal segment 66b. As used herein, the term “ray” differs from a geometric ray in that it may have a defined length similar to a line segment. A cone routine is executed for both the forward looking cone and the backward looking cone, as indicated by blocks 110 and 112 in FIG. 13. The cone routine returns a “maximum ray distance” (MRD) for a given cone. More specifically, and with reference to FIG. 14, the cone routine defines an opening angle alpha (a) of the cone pursuant to block 120, and defines under block 122 a number (n) of coaxial cones within the basic outer cone having respective opening angles αⱼ=j*α/n where j is an integer greater than zero and less than or equal to the number (n). The defined cones can be seen in FIG. 15. Then, the cone routine defines a polar array of points angularly spaced about the base circle of the cone by an angle beta (β) in accordance with block 124, which points can be seen in the view of FIG. 16. For each of these points, a ray of length SL/2 is generated in block 126. Then, in decision 128, the ray is analyzed to determine if it intersects the vessel boundary. If so, a ray distance (RD) is set as the length of the ray to the intersection point, as indicated by block 132. If the ray does not intersect the vessel boundary, flow proceeds to block 130 and the ray distance RD is set as the length SL/2 of the segment. Finally, in block 134, the maximum ray distance MRD is found from among all the ray distances RD, and this value is returned.

Thus, under blocks 110 and 112 in FIG. 13, a maximum ray distance for the forward looking (MRDF) and a maximum ray distance for the backward looking cone (MRDB) are found. Then, a maximum ray length (MRL) is calculated as:
MRL=(MRDF+MRDB)/SL
in accordance with block 114.

Continuing to block 116 in FIG. 13, a tortuousity value T associated with centerline point C is calculated as equal to (1−MRL)*1.5, wherein the value 1.5 can be thought of as a scale factor that may be chosen to suit a particular application. A colorwash function is carried out in block 118 to color a corresponding location on a digital image of the vessel based upon the local tortuousity value T. In the present example, the vessel location corresponding to centerline point C is colored green if T is less than 0.8, yellow if T is equal to or greater than 0.8 and less than 1.0, and red if T is equal to or greater than 1.0 and less than 1.5. As mentioned above, this step may be performed after T has been calculated for all centerline points C, or after each calculation of T as shown in FIG. 13.

Decision 119 determines if another centerline point C exists. If so, flow reverts to block 104 to repeat the procedure of positioning the stent and evaluating the stent position to determine tortuousity T at the next centerline point C. If there are no further centerline points C, the program is completed.

### EXAMPLE 4

### Three Joint Stent Along Vessel Centerline Path

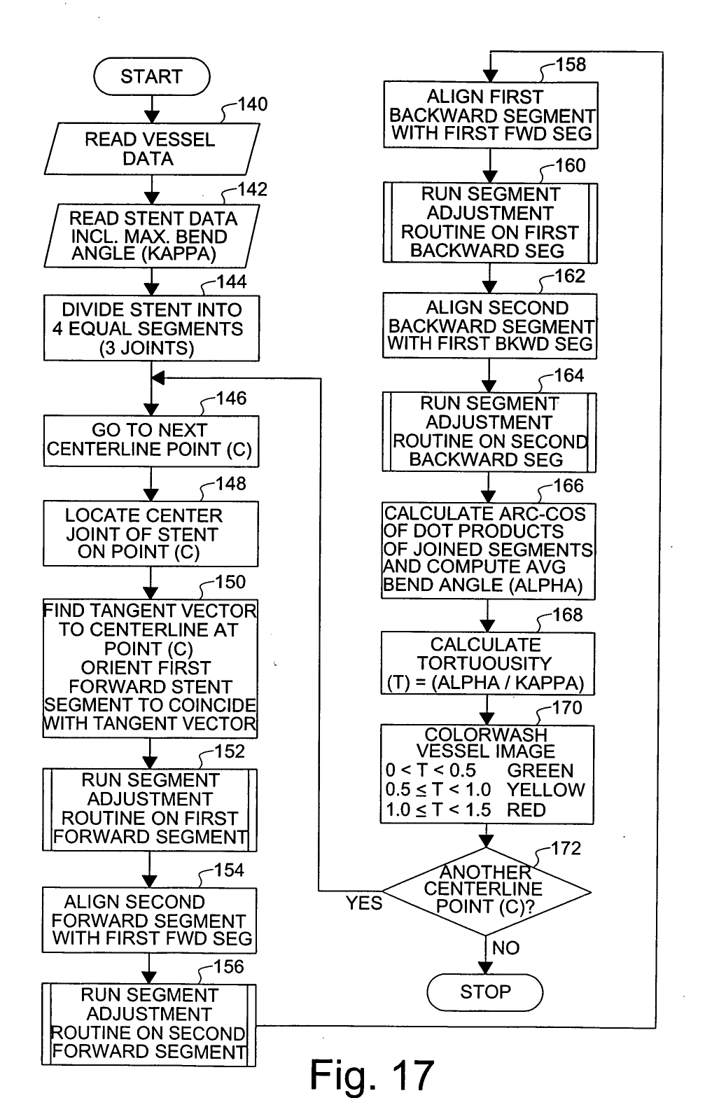

Reference is made to FIGS. 17-21 of the drawings. In this example, a stent 66 is assumed to comprise four segments 66a, 66b, 66c and 66d of equal length connected end-to-end by three joints 67, and the center joint 67 of the stent is always located on a centerline curve 64 extending through the vessel 60.

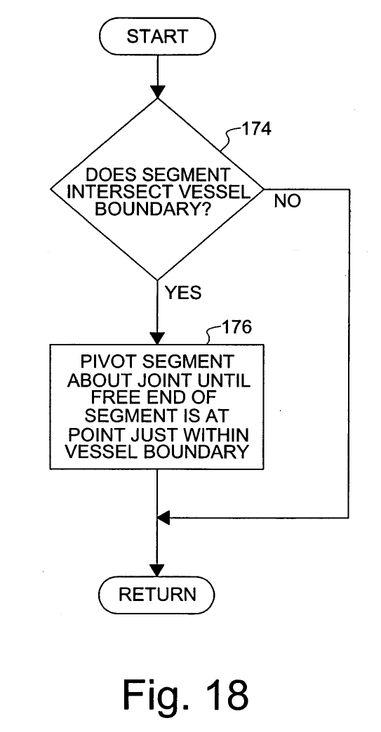

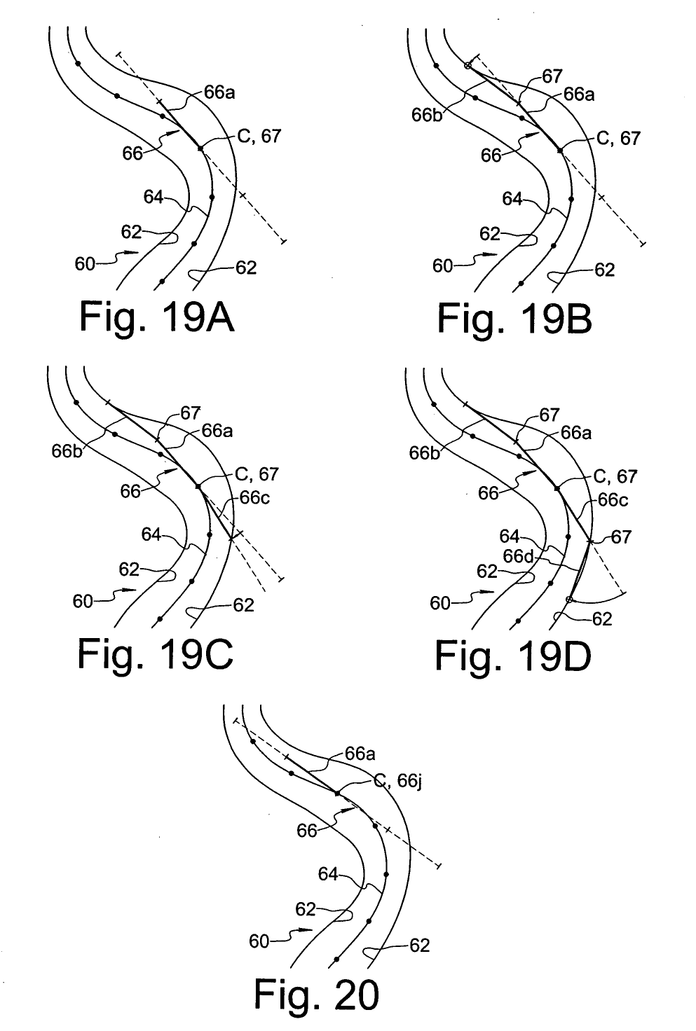

The flow diagram of FIG. 17 begins with blocks 140 and 142, wherein the vessel data and stent data are read as input data. As part of the stent data, a maximum bend angle (kappa) associated with joints 67 is assigned. Then, in block 144, the stent is divided into four equal segments 66a-66d by introducing joints 67 at one-quarter, midway, and three-quarters along the length of the stent, whereby the stent segments are assumed to be capable of pivoting about the centers of a joints 67 relative to one another as if connected by ball joints. The next vessel centerline point C (or initial point, if it is the first to be evaluated) is determined in block 146, and the center of the stent (that is, center joint 67) is located at centerline point C in accordance with block 148. Then, in block 150, the tangent vector to the centerline curve at point C is determined and the first forward segment 66a of the stent is pivoted about point C into alignment with the tangent vector direction. The orientation of segment 66a is shown at FIG. 19A. A segment adjustment routine is called in block 152 to adjust the orientation of segment 66a, if necessary. The segment adjustment routine is shown in FIG. 18 and begins with decision 174 to determine if the segment intersects the vessel boundary 62. If it does, orientation of the segment is adjusted in block 176 by pivoting the segment until a “free end” of the segment (the end of the segment farthest from centerline point C) is at a point just within or on the vessel boundary. This adjustment is illustrated in FIGS. 19B, 19C, and 19D for segments 66b, 66c, and 66d, respectively. If the segment does not intersect boundary 62, then adjustment step 176 is bypassed, as in the case of segment 66a.

Returning to FIG. 17, flow continues at block 154, whereby the second forward segment 66b is aligned with the first forward segment 66a. Then, in block 156, the segment adjustment routine is executed with respect to segment 66b. A similar procedure is followed for the first backward segment 66c pursuant to blocks 158 and 160. Then, the procedure is repeated under blocks 162 and 164 for the second backward segment 66d. As will be seen in FIG. 21, a series of bend angles alpha (a) associated with joints 67 result from the positioning steps illustrated in FIGS. 19A-19D. In step 166, the arc-cosine of the dot products of the joined segments 66a-66d is calculated and an average bend angle alpha is computed.

Proceeding to block 168, a tortuousity value T for centerline point C is calculated as (alpha)/(kappa). A colorwash function is carried out in block 170 to color a corresponding location on a digital image of the vessel based upon the local tortuousity value T. In the present example, the vessel location corresponding to centerline point C is colored green if T is less than 0.5, yellow if T is equal to or greater than 0.5 and less than 1.0, and red if T is equal to or greater than 1.0 and less than 1.5. As mentioned above, this step may be performed after T has been calculated for all centerline points C, or after each calculation of T as shown in FIG. 17.

Decision 172 determines if another centerline point C exists. If so, flow reverts to block 74 to repeat the procedure of positioning the stent 66 and evaluating the stent positions to determine tortuousity T. FIG. 20 shows advancement of the stent to a next location in the vessel according to the present example to begin the positioning procedure anew. If there are no further centerline points C, the program is completed.

### EXAMPLE 5

### Five Joint Stent at Boundary Limited Positions

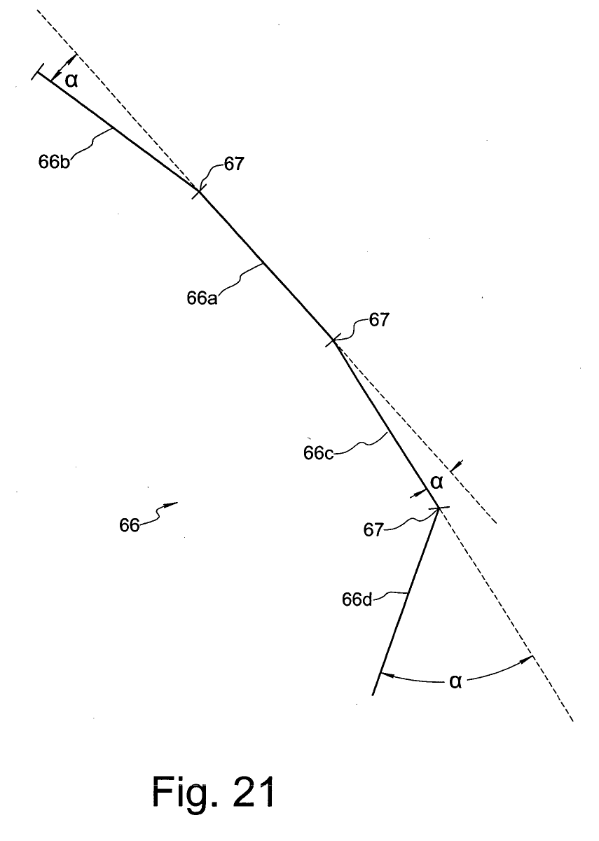

Reference is made to FIGS. 22-25 of the drawings. In this example, a stent is assumed to comprise six segments 66a, 66b, 66c, 66d, 66e, and 66f of equal length connected end-to-end by five joints 67. A proximal end of the stent is always located on a centerline curve 64 extending through the vessel 60, and the stent deforms according to the vessel boundary 62.

The flow diagram of FIG. 22 begins with blocks 180 and 182, wherein the vessel data and stent data are read as input data. As part of the stent data, a maximum bend angle (kappa) associated with joints 67 is assigned. Then, in block 184, the stent is divided into six equal segments 66a-66f by introducing joints 67 along the length of the stent, whereby the stent segments are assumed to be capable of pivoting about the centers of joints 67 relative to one another. A first or original vessel centerline point C is determined in block 186, and the proximal end of the first (most proximal) stent segment 66a is located at centerline point C in accordance with block 188. Then, in accordance with block 190, the first stent segment 66a is rotated about centerline point C to bring the segment into alignment with an estimated initial direction vector, which may be a tangent vector to centerline 64 at point C, or a local vector corresponding to a direction of the first several centerline points. Once the first segment 66a has been positioned, the segment adjustment routine of FIG. 18 is executed at block 192 to adjust the position of segment 66a if it intersects boundary 62.

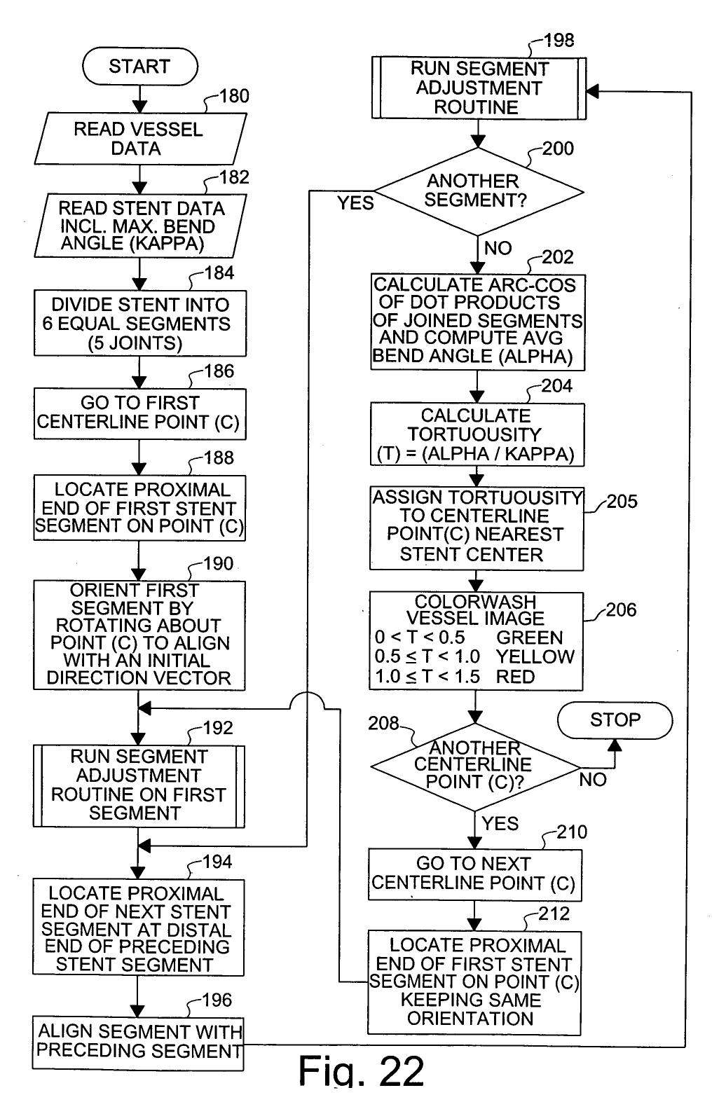

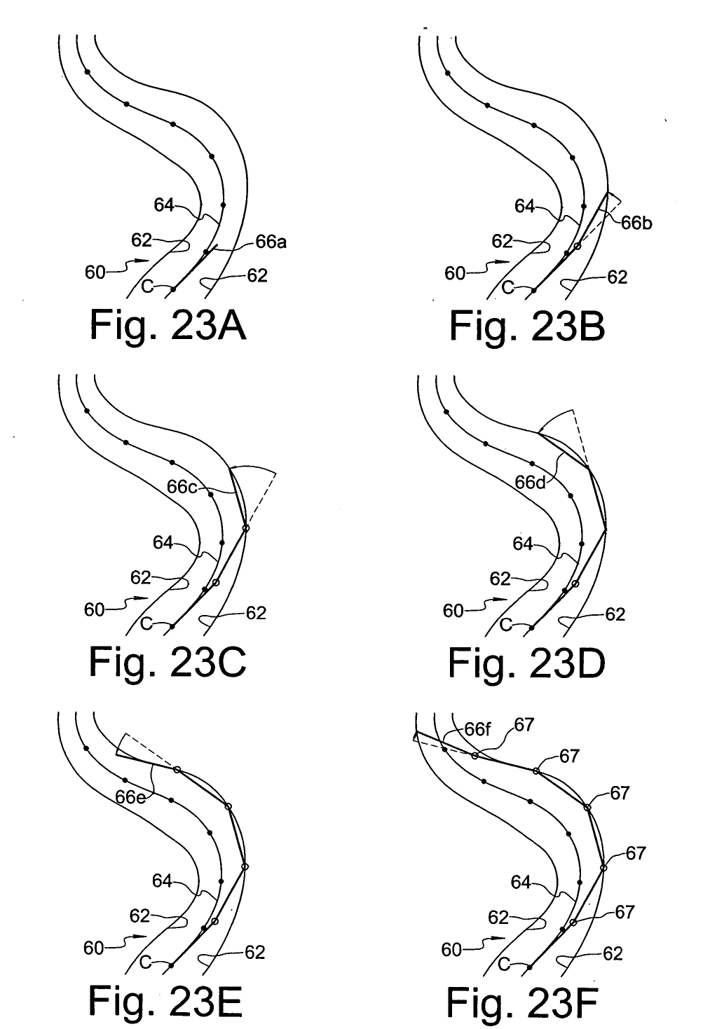

The subsequent segments 66b-66f are positioned sequentially in similar fashion, with the preceding segment determining the initial direction of each segment prior to any adjustment of the segment. Thus, in blocks 194 and 196, a next stent segment (e.g. second segment 66b) is located with its proximal end at the distal end of the preceding segment (e.g. first segment 66a), and is aligned with the preceding segment. Then, the adjustment routine is executed with respect to the newly located segment according to block 198. If there is another segment, decision 200 returns flow back to block 194 to position the next segment. The progression of segments can be seen in FIGS. 23A-23F. If all segments have been positioned, then the overall stent position has been modeled and decision 200 directs flow to block 202. In block 202, the arc-cosine of the dot products of the joined segments 66a-66f is calculated and an average bend angle alpha is computed.

Proceeding to block 204, a tortuousity value T is calculated as being equal to (alpha)/(kappa). However, this tortuousity value T is not assigned to the original centerline point C, but rather is assigned in block 205 to another centerline point C′ closest to the center joint 67 of the stent, as shown in FIG. 25. A colorwash function is carried out in block 206 to color a corresponding location on a digital image of the vessel based upon the local tortuousity value T. In the present example, the vessel location corresponding to centerline point C′ is colored green if T is less than 0.5, yellow if T is equal to or greater than 0.5 and less than 1.0, and red if T is equal to or greater than 1.0 and less than 1.5. As mentioned above, this step may be performed after T has been calculated for all positions of the stent, or after each calculation of T as shown in FIG. 22.

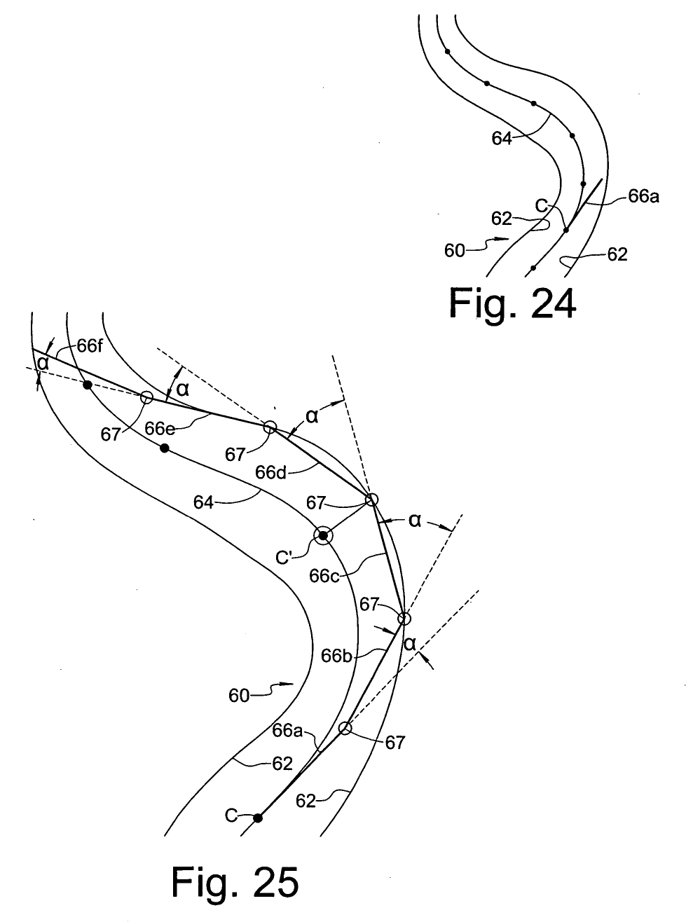

Following the colorwash step, decision 208 determines if another centerline point C exists. If so, the next centerline point C is found in block 210 and the proximal end of the first segment 66a is relocated to the next centerline point as indicated at block 212 and shown in FIG. 24, keeping the same orientation of the segment. Flow is returned to block 192 to again model a deformation position of the stent at the new location along vessel 60. If decision 208 determines there are no further centerline points for evaluation, then the program is completed.

### EXAMPLE 6

### Five Joint Stent Along Guidewire Path

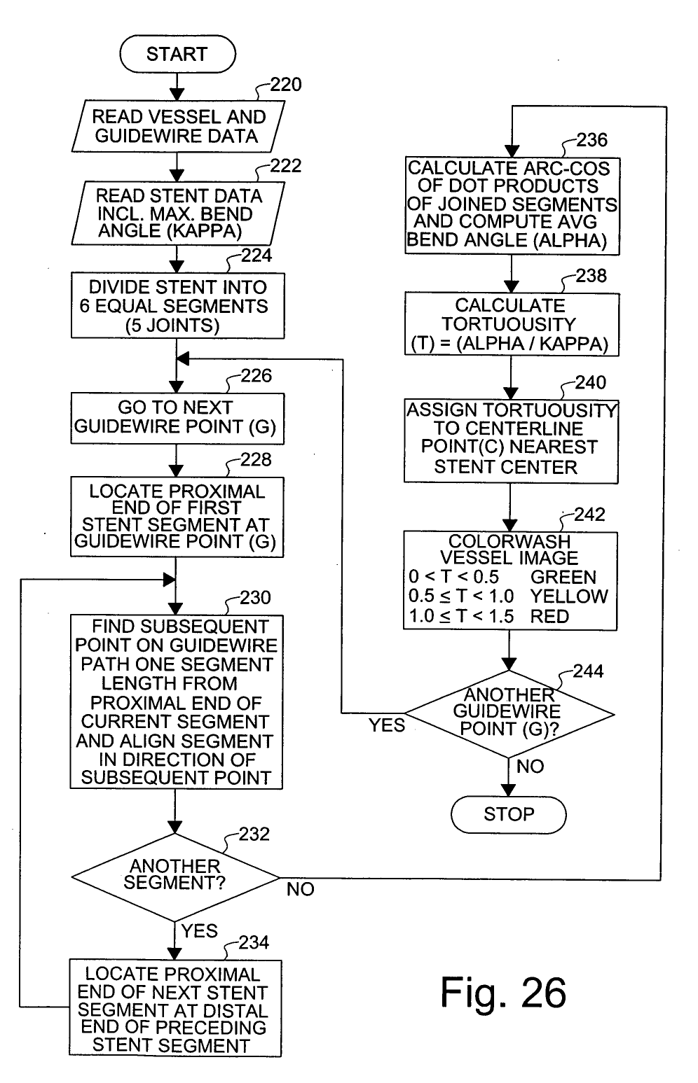

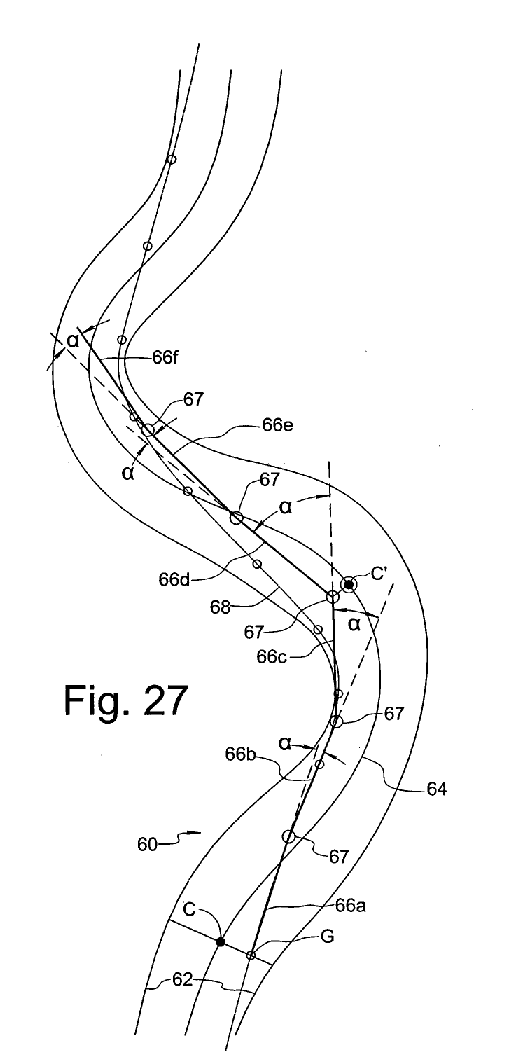

Reference is made to FIGS. 26 and 27 of the drawings. In this example, a stent is assumed to comprise six segments 66a, 66b, 66c, 66d, 66e, and 66f of equal length connected end-to-end by five joints 67. The stent segments are positioned approximately along a guidewire 68 inserted to extend through the vessel 60. The path of guidewire 68 may be represented by a parametric curve in three dimensions.

As shown in the flow diagram of FIG. 26, vessel data, guidewire data, and stent data, including the stent length, stent diameter, and a maximum bend angle (kappa) associated with joints 67 are read in blocks 220 and 222. Then, in block 224, the stent is divided into six equal segments 66a-66f by introducing joints 67 along the length of the stent, whereby the stent segments are assumed to be capable of pivoting about the centers of joints 67 relative to one another. The next guidewire point G (or initial point, if it is the first to be evaluated) is determined in block 226. The guidewire point G may be a point on guidewire 68 where the guidewire intersects a plane normal to centerline 64 at a corresponding centerline point C, as can be seen in FIG. 27. The proximal end of first stent segment 66a is located at guidewire point G as indicated by block 228. The stent segment 66a is then aligned toward a forward point on the guidewire one segment length from original guidewire point G according to block 230. Decision 232 determines if there is another segment to be positioned, and if so the proximal end of the next segment is positioned at the distal end of the preceding segment under block 234 and flow goes back to block 230. If there are no more segments, decision 232 directs flow to block 236. In block 236, the arc-cosine of the dot products of the joined segments 66a-66f is calculated and an average bend angle alpha is computed.

Proceeding to block 238, a tortuousity value T is calculated as being equal to (alpha)/(kappa). This tortuousity value T is not assigned to the original centerline point C, but rather is assigned in block 240 to another centerline point C′ closest to the center joint 67 of the stent, as shown in FIG. 27. A colorwash function is carried out in block 242 to color a corresponding location on a digital image of the vessel based upon the local tortuousity value T. In the present example, the vessel location corresponding to centerline point C′ is colored green if T is less than 0.5, yellow if T is equal to or greater than 0.5 and less than 1.0, and red if T is equal to or greater than 1.0 and less than 1.5. As mentioned above, this step may be performed after T has been calculated for all positions of the stent, or after each calculation of T as shown in FIG. 26.

After the colorwash step, decision 244 determines if another guidewire point G exists. If so, flow reverts to block 226 to advance the first stent segment to the next guidewire point. The next guidewire point may be a point in a direction corresponding to a local tangent of the guidewire at a distance that is the average distance between centerline points C. If there are no further guidewire points G, the program is completed.

### Other Example Embodiments

As will be appreciated from the foregoing example embodiments, there are of course other possible approaches to modeling a vessel and device. These include, without limitation, a rigid stent advancing along a guidewire path, a single jointed stent advancing along a guidewire path, and a three jointed stent advancing along a guidewire path.

What is claimed is:

1. A method for determining tortuousity of a vessel relative to a device to be moved through the vessel, the method comprising the steps of:
reading vessel data describing the vessel;
reading device data describing the device;
generating a model representing positions of the device along a travel path through the vessel based on the vessel data and the device data; and
evaluating the model to ascertain a degree to which the device protrudes from the travel path and/or deforms when the device is positioned at a corresponding point along the travel path.

2. The method according to claim 1, wherein the model is evaluated for a plurality of points each corresponding to a different position of the device along the travel path.

3. The method according to claim 1, wherein the vessel data describe a centerline curve through the vessel, and the travel path follows the centerline curve.

4. The method according to claim 1, further comprising the step of reading guidewire data describing a guidewire path through the vessel, wherein the travel path follows the guidewire path.

5. The method according to claim 1, wherein the device data include a length of the device.

6. The method according to claim 1, wherein the device data include a number of joined segments of the device.

7. The method according to claim 1, wherein the device is a stent.

8. The method according to claim 1, wherein the degree to which the device protrudes from the travel path and/or deforms is ascertained quantitatively.

9. The method according to claim 2, further comprising the step of reporting the degree.

10. The method according to claim 9, wherein the step of reporting includes generating a color-coded image of the vessel, wherein different colors in the image represent different degrees to which the device protrudes from the travel path and/or deforms.

11. A method for determining suitability of a stent for delivery through a vessel, the method comprising the steps of:
generating a model representing positions of the stent within the vessel; and
evaluating the model to ascertain a degree of risk associated with delivery of the stent through the vessel.

12. The method according to claim 11, further comprising the step of generating a color-coded image of the vessel for communicating the degree of risk.

13. A system for determining tortuousity of a vessel relative to a device to be moved through the vessel, the system comprising:
a computer system having stored thereon
programming code for generating a model representing positions of the device along a travel path through the vessel based on data describing the vessel and the device; and
programming code for evaluating the model to ascertain a degree to which the device protrudes from the travel path and/or deforms when the device is positioned at a corresponding point along the travel path.

14. The system according to claim 13, wherein the programming code for evaluating the model causes the model to be evaluated for a plurality of points each corresponding to a different position of the device along the travel path.

15. The system according to claim 13, wherein the data describing the vessel include data describing a centerline curve through the vessel, and the travel path follows the centerline curve.

16. The system according to claim 13, wherein the data further describe a guidewire path through the vessel, and the travel path follows the guidewire path.

17. The system according to claim 13, wherein the programming code for evaluating the model quantitatively ascertains the degree to which the device protrudes from the travel path and/or deforms.

18. The system according to claim 14, wherein the computer system includes a display and the computer system further has stored thereon programming code for reporting the degree on the display.

19. The system according to claim 18, wherein the programming code for reporting the degree generates a color-coded image of the vessel on the display, wherein different colors in the image represent different degrees to which the device protrudes from the travel path and/or deforms.
